(19) United States
(12) Patent Application Publication
(10) Pub. No.: US 2026/0132450 A1
(43) Pub. Date: May 14, 2026

(54) NUCLEIC ACID AMPLIFICATION METHOD

(71) Applicant: ANGLIA RUSKIN UNIVERSITY HIGHER EDUCATION CORPORATION, Essex (GB)

(72) Inventor: Stephen Bustin, Essex (GB)

(21) Appl. No.: 18/706,277

(22) PCT Filed: Oct. 27, 2022
(86) PCT No.: PCT/EP2022/080149

§ 371 Date: Apr. 30, 2024

(30) Foreign Application Priority Data
Nov. 1, 2021 (GB) 2115637.7

(51) Int. Cl. C12Q 1/6844; B01L 7/00; B01L 7/02

(52) U.S. Cl. CPC C12Q 1/6844; B01L 7/02; B01L 7/5255; B01L 2200/0647; B01L 2300/1838

(57) ABSTRACT

This invention relates to methods for amplifying target nucleic acid. A target nucleic acid is contacted with specific primers in the presence of DNA polymerase and deoxyribonucleotide triphosphates to produce an amplification solution with a volume of 5 pl or less. The amplification solution is subjected to a denaturation temperature of 75° C.-85° C. for 1.5 second or less; and then to a polymerisation temperature of 25° C.-45° C. for 1 second or less. These denaturation and polymerisation steps are repeated to amplify the target nucleic acid. Devices and systems for performing such methods are also provided.

### FIELD

The present invention relates to methods for nucleic acid amplification and devices for performing such methods.

### BACKGROUND

PCR is a molecular technology that amplifies specific sequences of DNA. Conventional two-step PCR methods are carried out using primers with melting temperatures (Tₘ) of around 60-65° C., generating PCR amplicons that are typically 100-200 bp long and have a Tₘ of greater than 80° C. Hence conventional PCR denaturation is carried out at 95° C., with the annealing/polymerisation step being run at 60-62° C. Typical reaction times are between 30 minutes and 1 hour and most reactions are carried out in 20-50 μL reaction volumes.

US2017327870A1 discloses compositions and methods for the amplification and analysis of nucleic acids which aim to minimise the generation of non-specific amplification products.

US2011136104A1 discloses one pot multiplexed quantitative PCR methods for end point analysis of a plurality of nucleic acid targets in a sample without user intervention, and to various encoded particles on which are immobilised one or more probes that hybridise with the plurality of targets.

CN107502657A discloses an extremely fast two-step PCR amplification and endpoint detection method that aims to complete and visually detect in a short time.

US2003087237A1 discloses methods for primer extension in low-temperature cycle DNA amplification using moderately thermostable DNA polymerases in the presence of a low concentration of glycerol or ethylene glycol as an agent to reduce the melting temperature of DNA.

Wheeler et al. (Analyst, 2011) describes PCR amplification of synthetic SARS respiratory pathogenic targets and bacterial genomic DNA in less than three minutes in which the sample is cycled between denaturation and annealing/extension temperatures of 94° C. and 55° C., respectively.

WO 2013/177429 A2 describes methods, devices, and kits for performing extreme PCR in which each cycle, comprising a denaturation step and annealing/extension step, is completed in less than 20 seconds per cycle. These methods use conventionally high denaturation and polymerisation temperatures.

Farrar and Wittwer (Clinical Chemistry, 2015) describes prototype PCR instruments which temperature cycle samples in 0.4-2.0 seconds at annealing/extension temperatures and denaturation temperatures of 62° C. to 76° C. and 85° C. to 92° C., respectively.

### SUMMARY

The present inventors have developed a method to rapidly amplify target nucleic acid at low temperatures and in a small reaction volume that allows, for example, the fast, convenient, and relatively low-cost amplification of target nucleic acid.

A first aspect of the invention provides a method for amplifying a target nucleic acid comprising:
(i) contacting a target nucleic acid with forward and reverse amplification primers specific for the target nucleic acid in the presence of DNA polymerase and deoxyribonucleotide triphosphates to produce an amplification solution,
   wherein the amplification solution has a volume of 5 μl or less,
(ii) subjecting the amplification solution to a denaturation temperature of 75° C.-85° C. for 1.5 second or less, such that nucleic acid in the amplification solution is denatured,
(iii) subjecting the amplification solution to a polymerisation temperature of 25° C.-45° C. for 1 second or less,
   such that the primers hybridise to the target nucleic acid and are extended by the polymerase,
(iv) repeating steps (ii) and (iii) one or more times, thereby amplifying the target nucleic acid.

In some embodiments of methods of the first aspect, the target nucleic acid is comprised within a sample nucleic acid. The sample nucleic acid may be a deoxyribonucleic acid. Suitable sample nucleic acid may be produced by a method comprising providing a sample ribonucleic acid and reverse transcribing the sample ribonucleic acid to produce the sample nucleic acid.

A second aspect of the invention provides a nucleic acid amplification device comprising;
a denaturation region,
a polymerisation region,
a heater to heat the denaturation region to between 75° C.-85° C.,
a heater to heat the polymerisation region to between 25° C.-45° C., and
an actuator to move an amplification vessel for containing an amplification solution between the denaturation region and the polymerisation region.

The device may be configured for amplifying a target nucleic acid by a method of the first aspect. For example, the device may further comprise a processor programmed to operate the device to amplify a target nucleic acid by a method of the first aspect.

A third aspect of the invention provides a system for amplifying a target nucleic acid comprising:
(i) a device of the second aspect of the invention;
(ii) a forward amplification primer and a reverse amplification primer which are specific for the target nucleic acid;
(iii) deoxyribonucleotide triphosphates and;
(iv) a DNA polymerase.

A kit of the third aspect may be suitable for use in a method of the first aspect.

Other aspects and embodiments of the invention are described in more detail below.

### BRIEF DESCRIPTION OF THE FIGURES

FIG. 1 shows the bespoke PCR instrument (“Mk1extreme PCR device”) designed by the present inventors, which comprises two water baths and a robotic arm capable of moving a plate or tubes between the two water baths at adjustable speeds.

FIG. 2 shows the results of qPCR using a sample pre-amplified by a 20-cycle amplification performed on the Mk1extreme PCR device using optimised assay conditions (brown), a sample pre-amplified by a 20-cycle amplification performed on the Mk1extreme PCR device using non-optimised assay conditions (blue), and a sample using non-optimised assay conditions which was not pre-amplified (red). (A) shows an amplification plot and (B) shows the Quantification cycle (Cq) for each sample.

FIG. 3 shows the results of a qPCR using a sample pre-amplified by a 10-cycle amplification performed on the Mk1extreme PCR device probing for the E484 WT sequence (blue) or E-gene (green). (A) shows an amplification plot and (B) shows the Cq for each sample.

FIG. 4 shows the PCR program (upper panels) and Cq results (lower panels) of qPCR reactions probing for 12 different targets performed on a standard qPCR instrument at denaturation temperatures ranging from 80-95° C. (A) and 75-85° C. (B). (C) plots the change in Cq between 80-95° C. (upper) and 75-85° C. (lower) for each qPCR reaction.

FIG. 5A shows the PCR program (upper panel) and Cq results (lower panel) of qPCR reactions probing for 12 different targets performed on a standard qPCR instrument at denaturation temperatures ranging from 65.0-80.5° C. FIG. 5B plots the Cq at temperatures between 79-81.5° C. (upper) and the change in Cq between 79-85° C. (B) for each qPCR reaction.

FIG. 6 shows the PCR program (upper panels) and Cq results (lower panels) of qPCR reactions probing for 12 different targets performed on a standard qPCR instrument at polymerisation temperatures ranging from 30-54° C. (A) and 46-63° C. (B). (C) plots the change in Cq between 30-54° C. (upper) and 46-63° C. (lower) for each qPCR reaction.

### DETAILED DESCRIPTION

This invention relates to a method for amplifying a target nucleic acid that comprises contacting a target nucleic acid with a forward amplification primer and a reverse amplification primer in the presence of DNA polymerase and deoxyribonucleotide triphosphates to produce an amplification solution. The forward amplification primer and the reverse amplification primer are specific for the target nucleic acid. The amplification solution has a volume of 5 μl or less. The amplification solution is then subjected to a denaturation temperature of 75° C.-85° C. for 1.5 second or less, such that nucleic acid in the amplification solution is denatured, followed by a polymerisation temperature of 25° C.-45° C. for 1 second or less, such that the primers hybridise to the target nucleic acid and are extended by the polymerase. These steps are repeated one or more times to amplify the target nucleic acid.

The methods described herein may allow, for example, the rapid amplification and detection of a target nucleic acid and may be suitable, for example, for the diagnosis of a viral infection using a point-of-care nucleic acid amplification device. Nucleic acid amplification devices configured for the methods described herein use lower temperatures and may have reduced power consumption than standard nucleic acid amplification devices. This may reduce running costs and may be particularly advantageous for point-of-care amplification devices, which may be battery operated.

The methods described herein may amplify nucleic acid through the polymerase chain reaction (PCR). The amplification of nucleic acid using the polymerase chain reaction is well known in the art.

The target nucleic acid is amplified as described herein in an amplification solution. An amplification solution is a reaction mixture that supports the amplification of nucleic acids. Suitable amplification solutions may, for example, contain a target nucleic acid, a forward amplification primer, a reverse amplification primer, a DNA polymerase and deoxyribonucleotide triphosphates.

A nucleic acid may be a naturally occurring or synthetic oligonucleotide or polynucleotide. Nucleic acids have a 5′ end and a 3′ end. The nucleotide at the 5′ end of a nucleic acid has a carbon at the fifth position (5′ carbon) in the sugar ring (e.g. deoxyribose or ribose) that is not linked to further nucleotides. The 5′ carbon may, for example, be attached to a phosphate group. The nucleotide at the 3′end of a nucleic acid has a carbon at the third position (3′ carbon) in the sugar ring (e.g. deoxyribose or ribose) that is not linked to further nucleotides. The 3′ carbon may for example be attached to a hydroxyl group. Nucleic acids described herein may be double-stranded or single-stranded.

The methods described herein amplify a target nucleic acid i.e. the number of copies of target nucleic acid is increased following amplification. The target nucleic acid is composed of a sequence of nucleotides (which may be referred to herein as the target nucleic acid sequence). In some embodiments, the target nucleic acid may be single-stranded. For example, the target nucleic acid may be a portion of the genome of a ssDNA virus or the product of a reverse transcription reaction (for example, the DNA sequence produced after reverse transcription of mRNA). In other embodiments, the target nucleic acid may be double-stranded. For example, the target nucleic may be a portion of a double stranded genome, for example a fungal or bacterial genome. A double-stranded target nucleic acid may comprise a sense strand and an antisense strand. The sense and antisense strands may be complementary. The sequence of the target nucleic acid may be determined from the sense strand and/or the antisense strand.

In some embodiments, the target nucleic acid may be present within a sample nucleic acid. The sample nucleic acid may comprise the target nucleic acid and additional nucleic acid. The additional nucleic acid may for example flank the target nucleic acid at one or both ends. In some embodiments, the target nucleic acid may be located within the sample nucleic acid. Suitable sample nucleic acid may include, for example, a genome or a fragment thereof. For example, the sample nucleic acid may be a viral genome comprising the target nucleic acid, a bacterial genome comprising the target nucleic acid, or a fungal genome comprising the target nucleic acid.

In some embodiments, the sample nucleic acid may be present in a sample, for example a sample previously obtained from an individual. The sample obtained from an individual may be, for example, a bodily fluid such as saliva, mucus, or blood.

Primers are oligonucleotides that prime DNA synthesis by a template-dependent DNA polymerase. A primer hybridises to an extremity of the target nucleic acid in a sequence-specific manner to permit selective amplification of the target nucleic acid. The 3′ end of the primer provides a 3′-OH group to which further nucleotides may be attached by a template-dependent DNA polymerase establishing 3′- to 5′-phosphodiester linkage, whereby deoxyribonucleotide triphosphates are incorporated into the growing DNA strand and pyrophosphate is released.

Nucleic acid amplification is performed using a forward amplification primer and reverse amplification primer. The forward amplification primer primes DNA synthesis in the forward direction (corresponding, for example, to the direction from the 5′ end to the 3′ end of the target nucleic acid on the sense strand of a dsDNA molecule). The reverse amplification primer primes DNA synthesis in the reverse direction (corresponding, for example, to the direction from the 3′ end to the 5′ end of the sense strand of a dsDNA molecule).

For example, for a dsDNA target nucleic acid, the forward amplification primer may hybridise to a nucleotide sequence at the 3′ end of the antisense strand of the target nucleic acid. The 3′ end of the forward amplification primer provides a 3′-OH group to which further nucleotides may be attached by a template-dependent DNA polymerase to extend the growing DNA strand in the forward direction along the antisense strand template (i.e. to extend a nascent sense strand in the 5′ to 3′ direction). Similarly, the reverse amplification primer may hybridise to a nucleotide sequence at the 3′ end of the sense strand of the target nucleic acid. The 3′end of the reverse amplification primer provides a 3′-OH group to which further nucleotides may be attached by a template-dependent DNA polymerase in order to extend the growing DNA strand in the reverse direction along the sense strand template (i.e. to extend a nascent antisense strand in the 5′ to 3′ direction).

Preferred primers may be single-stranded.

The amplification solution may further comprise a probe to determine the amount of amplified target nucleic acid. A probe is an oligonucleotide which serves to detect amplified target nucleic acid. For example, a probe may be an oligonucleotide which can hybridise to the target nucleic acid, or a portion thereof, and which is detectably labelled. For example, a probe may be radioactively, fluorescently, or non-radioactively labelled. Preferably, the probe is fluorescently labelled.

Binding of the probe to the amplified target nucleic acid, or a portion thereof, may cause emission of the detectable signal. The amount of detectable signal emitted may be quantified and compared to, for example, a standard curve that correlates the signal generated by binding of a target nucleic acid, or a portion thereof, to the corresponding probe over a broad range of target concentrations or an endogenous reference gene such as, for example, beta-actin. Accordingly, the probe can be used to determine the amount of amplified target nucleic acid.

Preferably, the probe targets the same strand of the target nucleic acid as the forward amplification primer. For example, the 5′ end of the probe may bind to the strand (such as, for example, the antisense strand of a dsDNA molecule) 1 or 2 nucleotides away from the 3′ end of the forward amplification primer. By allowing the 3′ end of the reverse amplification primer to overlap with the 3′ end or the probe (for example, overlap between the three bases at the 3′ end of the reverse amplification primer and the three bases at the 3′ end of the probe three bases at the 3′ end of the probe), the length of the target nucleic acid can be minimised. However, the longer the overlap between the 3′ ends of the reverse amplification primer and the probe the higher the risk of the reverse amplification primer and the probe hybridising to each other, thereby interfering with detection of amplified target nucleic acid and/or decreasing the efficiency of the amplification reaction. Preferably, the overlap between the 3′ ends of the reverse amplification primer and the probe is 5 bases or fewer, for example 4 bases, 3 bases, 2 bases, or 1 base.

In some embodiments, the probe may comprise a modified nucleoside. For example, in some embodiments, the probe includes one or more locked nucleic acid (LNA) monomers, in which a nucleic acid monomer comprises a 2′-O,4′C bridge which locks the structure into a bicyclic formation. The melting temperature of a complex in which the target nucleic acid is hybridised to a probe comprising one or more locked nucleic acid monomers (an LNA probe) is higher than the melting temperature of the target nucleic acid. Accordingly, LNA probes are less tolerant of mismatches and so, relative to “conventional” unmodified nucleic acid probes, a shorter probe can be used whilst maintaining specificity.

In some embodiments, the 3′ end of the probe may be modified (e.g. phosphorylated, or to include an inverted dT or dideoxycytidine (ddC)) to prevent any potential unwanted extension of the probe.

A method described herein may comprise identifying a suitable target nucleic acid. Preferable characteristics of a target nucleic acid are disclosed herein. For example, a target nucleic acid with a low melting temperature is particularly suitable for the amplification method disclosed herein. One way to achieve a low melting temperature this is to minimise the length of the target nucleic acid.

A target nucleic acid suitable for amplification by the methods described herein may be identified using methods established in the art. For example, a nucleotide sequence that is unique for a target pathogen may be identified with a BLAST search using a target gene (e.g. 28S rRNA). Specific sequences within the target gene may then be identified which distinguish the target from closely related targets, for example distinguishing Candida auris from Candida albicans. Several such distinguishing sequences may be re-analysed by BLAST and one or more target nucleic acids identified by selecting a target nucleic acid from amongst the distinguishing sequences that has the properties (such as a low melting temperature) described herein. Preferred target nucleic acids may be short and/or display minimal secondary structure.

After identification of the target nucleic acid, the amplification conditions may be optimised by designing primers and probes. Preferable characteristics of primers and probes are disclosed herein. Numerous methods of designing primers and probes suitable for use in the amplification methods disclosed herein are available in the art. For example, primers may be designed using design software (such as BeaconDesigner (AleleID)) and manually adjusted to fit the 3′ ends of the primers into the target nucleic acid identified using BLAST. The specificity of the design can be analysed using software (such as PrimerBLAST (NCBI)) and, if necessary, further adjustments can be made to the primers, for instance to shorten or lengthen the primer. After identification of the target nucleic acid and forward and reverse amplification primers, a probe may be designed which can hybridise to the target nucleic acid, or a portion thereof.

Numerous primer design tools are available for designing amplification primers, such as Primer-BLAST (NCBI), Applied Biosystems™ Primer Designer™ Tool (ThermoFisher), Prime+ (Eurofins Genomics) and BeaconDesigner (AleleID). Suitable primers, including custom-synthesised primers, may be synthesised using known methods or obtained from commercial suppliers, such as Sigma Aldrich, Eurofins Genomics, ThermoFisher and Integrated DNA Technologies.

Numerous probe design tools are available for designing the probes, such as RealTime PCR Design Tool (Integrated DNA Technologies) and qPCR Primer & Probe Design Tool (Eurofins Genomics). Suitable probes, including custom-designed probes, may be synthesised using known methods or obtained from commercial suppliers, such as ThermoFisher, Integrated DNA Technologies, BioLegio and Eurogentec.

The melting temperature or “Tₘ” of a nucleic acid is the temperature at which the single-stranded and double-stranded forms of a nucleic acid exist in equilibrium. This may depend upon many factors, including, for example, the length and nucleotide sequence of the nucleic acid.

The melting temperature of a target nucleic acid is the temperature at which the double-stranded form of the target nucleic acid is converted to the single-stranded form of the target nucleic acid. Preferably, the melting temperature of the target nucleic acid is between 65° C. and 75° C. For example, the melting temperature of the target nucleic acid may be around 65° C., around 66° C., around 67° C., around 68° C., around 69° C., around 70° C., around 71° C., around 72° C., around 73° C., around 74° C., or around 75° C. For example, the melting temperature of the target nucleic acid may be 65° C., 66° C., 67° C., 68° C., 69° C., 70° C., 71° C., 72° C., 73° C., 74° C., or 75° C. Most preferably, the melting temperature of the target nucleic acid is around 70° C., for example the melting temperature of the target nucleic acid may be 70° C. A “low” melting temperature of a target nucleic acid refers to, for example, a melting temperature which is lower than or equal to around 75° C.

Preferably, the forward amplification primer has a melting temperature of between 40° C. to 50° C. For example, the forward amplification primer may have a melting temperature of around 40° C., around 41° C., around 42° C., around 43° C., around 44° C., around 45° C., around 46° C., around 47° C., around 48° C., around 49° C., or around 50° C. More preferably, the forward amplification primer may have a melting temperature of 40° C., 41° C., 42° C., 43° C., 44° C., 45° C., 46° C., 47° C., 48° C., 49° C., or 50° C. Most preferably, the forward amplification primer has a melting temperature of around 45° C., for example the forward amplification primer may have a melting temperature of 45° C. A “low” melting temperature of a forward amplification primer refers to, for example, a melting temperature which is lower than or equal to around 50° C.

Preferably, the reverse amplification primer has a melting temperature of between 40° C. to 50° C. For example, the reverse amplification primer may have a melting temperature of around 40° C., around 41° C., around 42° C., around 43° C., around 44° C., around 45° C., around 46° C., around 47° C., around 48° C., around 49° C., or around 50° C. More preferably, the reverse amplification primer may have a melting temperature of 40° C., 41° C., 42° C., 43° C., 44° C., 45° C., 46° C., 47° C., 48° C., 49° C., or 50° C. Most preferably, the reverse amplification primer has a melting temperature of around 45° C., for example the reverse amplification primer may have a melting temperature of 45° C. A “low” melting temperature of a reverse amplification primer refers to, for example, a melting temperature which is lower than or equal to around 50° C.

In some embodiments, the amplification solution may comprise one or more PCR enhancers. PCR enhancers may reduce the melting temperature of the target nucleic acid. Suitable PCR enhancers are known in the art and may include Magnesium ions, Potassium ions, Betaine, DMSO, tetramethyl ammonium chloride (TMAC) and formamide.

Optimisation of the target nucleic acid may provide a target nucleic acid having a melting temperature of between 65° C. and 75° C. or any other melting temperature of the target nucleic acid described above. Preferably, optimisation of the target nucleic acid provides a target nucleic acid having a melting temperature of 70° C. These target nucleic acid melting temperatures are reduced as compared to the target nucleic acid melting temperatures used in conventional two-step PCR methods.

In other embodiments, the amplification solution may lack a PCR enhancer, or may contain a PCR enhancer in an amount that is too low to exert a PCR-enhancing effect, for example a melting temperature-reducing effect. For example, the amplification solution may lack a PCR enhancer which is an organic compound, or may contain a PCR enhancer that is an organic compound in an amount that is too low to exert a PCR-enhancing effect, for example a melting temperature-reducing effect. Preferably, the amplification solution lacks glycerol and/or ethylene glycol or contains glycerol and/or ethylene glycol in an amount that is too low to exert a PCR-enhancing effect, for example a melting temperature-reducing effect.

The melting temperatures of the forward amplification primer, reverse amplification primer, and target nucleic acid are preferably low in the amplification method disclosed herein. One way of achieving this is keeping the forward amplification primer, reverse amplification primer and target nucleic acid as short as possible.

In some embodiments, the target nucleic acid may be 45-65 bases in length. Preferably, the target nucleic acid is 50-60 bases in length. For example, the target nucleic acid may comprise 50 bases, 51 bases, 52 bases, 53 bases, 54 bases, 55 bases, 56 bases, 57 bases, 58 bases, 59 bases, or 60 bases in length. A “short” target nucleic acid is, for example, less than or equal to 65 bases in length.

In some embodiments, the primer may be 10-30 bases in length. Preferably, the primer is 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases in length. Preferably, the primer is 15-25 bases in length, for example, the primer is 15 bases, 16 bases, 17 bases, 18 bases, 19 bases, 20 bases, 21 bases, 22 bases, 23 bases, 24 bases, or 25 bases in length. Most preferably, the primer is 15-20 bases in length, for example, the probe is 15 bases, 16 bases, 17 bases, 18 bases, 19 bases, or 20 bases in length. A “short” primer is, for example, less than or equal to 25 bases in length.

In some embodiments, the probe may be 10-30 bases in length. Preferably, the probe is 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, or 30 bases in length. Preferably, the probe is 15-25 bases in length, for example, the probe is 15 bases, 16 bases, 17 bases, 18 bases, 19 bases, 20 bases, 21 bases, 22 bases, 23 bases, 24 bases, or 25 bases in length. Most preferably, the probe is 15-20 bases in length, for example, the probe is 15 bases, 16 bases, 17 bases, 18 bases, 19 bases, or 20 bases in length. A “short” probe is, for example, less than or equal to 25 bases in length.

The amplification method disclosed herein is particularly well suited for the amplification of AT-rich target nucleic acids. The DNA of bacteria and nucleic acid of viruses is typically AT-rich, so the methods disclosed herein may find use in the amplification of target bacterial and/or viral nucleic acid sequences. However, the method can also be used with more GC-rich target nucleic acids, such as fungal DNA sequences, if the assays are appropriately designed. For example, the assay could be designed to target the least GC-rich region possible in a fungal DNA sequence. Alternatively, or in addition, a GC-rich DNA target nucleic acid could be amplified using the amplification method disclosed herein using shorter primers and/or higher denaturation temperatures than for AT-rich target nucleic acid and/or LNA probes. Alternatively, or in addition, where the chosen primers result in reduced sensitivity (for example, due to the use of shorter primers), this could be mitigated by designing three or more separate assays detected by the same detectable label, for example the same fluorophore.

In some embodiments, the % AT content for the target nucleic acid may be between 40% and 60%. In some embodiments, the % AT content for the target nucleic acid may be around 40%, around 41%, around 42%, around 43%, around 44%, around 45%, around 46%, around 47%, around 48%, around 49%, around 50%, around 51%, around 52%, around 53%, around 54%, around 55%, around 56%, around 57%, around 58%, around 59%, or around 60%. For example, the % AT content for the target nucleic acid may be 40%, 41%, 42%, 43%, 44%, 45%, 46%, 47%, 48%, 49%, 50%, 51%, 52%, 53%, 54%, 55%, 56%, 57%, 58%, 59%, or 60%. Preferably, the % AT content for the target nucleic acid may be between 50% and 60%. In some preferred embodiments, the % AT content for the target nucleic acid may be around 50%, around 51%, around 52%, around 53%, around 54%, around 55%, around 56%, around 57%, around 58%, around 59%, or around 60%, for example, the % AT content for the target nucleic acid may be 50%, 51%, 52%, 53%, 54%, 55%, 56%, 57%, 58%, 59%, or 60%

In some embodiments, the % AT content for the forward amplification primer may be between 40% and 60%. In some embodiments, the % AT content for the forward amplification primer may be around 40%, around 41%, around 42%, around 43%, around 44%, around 45%, around 46%, around 47%, around 48%, around 49%, around 50%, around 51%, around 52%, around 53%, around 54%, around 55%, around 56%, around 57%, around 58%, around 59%, or around 60%. For example, the % AT content for the forward amplification primer may be 40%, 41%, 42%, 43%, 44%, 45%, 46%, 47%, 48%, 49%, 50%, 51%, 52%, 53%, 54%, 55%, 56%, 57%, 58%, 59%, or 60%. Preferably, the % AT content for the forward amplification primer may be between 50% and 60%. In some preferred embodiments, the % AT content for the forward amplification primer may be around 50%, around 51%, around 52%, around 53%, around 54%, around 55%, around 56%, around 57%, around 58%, around 59%, or around 60%, for example, the % AT content for the forward amplification primer may be 50%, 51%, 52%, 53%, 54%, 55%, 56%, 57%, 58%, 59%, or 60%

In some embodiments, the % AT content for the reverse amplification primer may be between 40% and 60%. In some embodiments, the % AT content for the reverse amplification primer may be around 40%, around 41%, around 42%, around 43%, around 44%, around 45%, around 46%, around 47%, around 48%, around 49%, around 50%, around 51%, around 52%, around 53%, around 54%, around 55%, around 56%, around 57%, around 58%, around 59%, or around 60%. For example, the % AT content for the reverse amplification primer may be 40%, 41%, 42%, 43%, 44%, 45%, 46%, 47%, 48%, 49%, 50%, 51%, 52%, 53%, 54%, 55%, 56%, 57%, 58%, 59%, or 60%. Preferably, the % AT content for the reverse amplification primer may be between 50% and 60%. In some preferred embodiments, the % AT content for the reverse amplification primer may be around 50%, around 51%, around 52%, around 53%, around 54%, around 55%, around 56%, around 57%, around 58%, around 59%, or around 60%, for example, the % AT content for the reverse amplification primer may be 50%, 51%, 52%, 53%, 54%, 55%, 56%, 57%, 58%, 59%, or 60%

A DNA polymerase catalyses the template-dependent synthesis of DNA molecules from deoxyribonucleotide triphosphates. The DNA polymerase catalyses the formation of primer extension products complementary to a template. The DNA polymerase achieves this by attaching further nucleotides to the 3′ end of a primer hybridised to the target nucleic acid.

Preferably, the DNA polymerase is a thermostable polymerase. Preferably, the DNA polymerase is a Taq polymerase. For example, the DNA polymerase may be KlenTaq polymerase. Suitable Taq polymerases are available from commercial suppliers, including Bioline, Takara, Sigma Aldrich, ThermoFisher, Promega and Qiagen.

Deoxyribonucleotide triphosphates may include dATP, dCTP, dGTP and dTTP. Deoxyribonucleotide triphosphates are used by DNA polymerase to synthesise DNA molecules.

The amplification reactions described herein are performed in a small volume. Preferably, the volume of the amplification solution is around 5 μL or less, around 4.5 μL or less, around 4 μL or less, around 3.5 μL or less, around 3 μL or less, around 2.5 μL or less, around 2 μL or less, around 1.5 μL or less, around 1 μL or less, around 0.75 μL or less, around 0.5 μL or less, around 0.25 μL or less, around 0.2 μL or less, or around 0.1 μL or less. More preferably, the volume of the amplification solution is 5 μL or less, 4.5 μL or less, 4 μL or less, 3.5 μL or less, 3 μL or less, 2.5 μL or less, 2 μL or less, 1.5 μL or less, 1 μL or less, 0.75 μL or less, 0.5 μL or less, 0.25 μL or less, 0.2 μL or less, or 0.1 μL or less. A “low” amplification volume is, for example, less than or equal to around 5 μL.

Preferably, the concentration of the forward amplification primer in the amplification solution is between 5 μM and 15 μM. For example, the concentration of the forward amplification primer in the amplification solution may be between 7.5 μM and 12.5 μM, 8 μM to 12 μM, or 9 μM to 11 μM. More preferably, the concentration of the forward amplification primer in the amplification solution is around 10 μM, for example the concentration of the forward amplification primer in the amplification solution may be 10 μM. A “high” concentration of forward amplification primer in the amplification solution is, for example, greater than or equal to 5 μM.

Preferably, the concentration of the reverse amplification primer in the amplification solution is between 5 μM and 15 μM. For example, the concentration of the reverse amplification primer in the amplification solution may be between 7.5 μM and 12.5 μM, 8 μM to 12 μM, or 9 μM to 11 μM. More preferably, the concentration of the reverse amplification primer in the amplification solution is around 10 μM, for example the concentration of the reverse amplification primer in the amplification solution may be 10 μM. A “high” concentration of reverse amplification primer in the amplification solution is, for example, greater than or equal to 5 μM.

Preferably, the concentration of the probe in the amplification solution is between 100 nM-200 nM. More preferably, the concentration of the probe in the amplification solution is around 100 nM, around 110 nM, around 120 nM, around 130 nM, around 140 nM, around 150 nM, around 160 nM, around 170 nM, around 180 nM, around 190 nM, or around 200 nM. More preferably, the concentration of the probe in the amplification solution is 100 nM, 110 nM, 120 nM, 130 nM, 140 nM, 150 nM, 160 nM, 170 nM, 180 nM, 190 nM, or 200 nM.

In some embodiments, the concentration of the DNA polymerase in the amplification solution is between 10 U/μL to 100 U/μL. Preferably, the concentration of the DNA polymerase around 10 U/μL, around 20 U/μL, around 30 U/μL, around 40 U/μL, around 50 U/μL, around 60 U/μL, around 70 U/μL, around 80 U/L, around 90 U/μL, or around 100 U/μL, for example, the concentration of the DNA polymerase is 10 U/μL, 20 U/μL, 30 U/μL, 40 U/μL, 50 U/μL, 60 U/μL, 70 U/μL, 80 U/μL, 90 U/μL, or 100 U/μL. More preferably, the concentration of the DNA polymerase is between 30 U/μL to 100 U/μL, 40 U/μL to 100 U/μL, 50 U/μL to 100 U/μL, 60 U/μL to 100 U/μL, 70 U/μL to 100 U/μL, 80 U/μL to 100 U/μL, or 90 U/μL to 100 U/μL. Most preferably, the concentration of the DNA polymerase is around 50 U/μL, for example the concentration of the DNA polymerase is 50 U/μL.

The amplification method disclosed herein is a two-step polymerase chain reaction (PCR) method comprising a denaturation step and a polymerisation step.

Double-stranded nucleic acid may be denatured by physical, chemical or enzymatic means to separate the strands of the double stranded molecule into single strands. For example, double-stranded nucleic acid may be denatured in the methods described herein by heating the double-stranded nucleic acid until it is predominantly or completely denatured (e.g., greater than 50%, 60%, 70%, 80%, 90%, or 95% denatured).

Denaturation of the target nucleic acid for example occurs when the amplification solution is subjected to the denaturation temperature.

Without being bound by theory, in a two-step PCR method, the amplification solution is subjected to a polymerisation temperature at which the forward and reverse amplification primers hybridise to the target nucleic acid and at which the extension of new DNA strands from the annealed primers is expected to occur. In other words, the hybridisation step and the extension step of three-step PCR occur at the polymerisation temperature. By removing the need for a temperature adjustment between a separate annealing and extension temperature, two-step PCR allows PCR reactions to be performed more quickly than three-step PCR.

Polymerisation may include the extension by a template-dependent DNA polymerase of a primer along a template sequence to form a nucleic acid strand that is complementary to the template sequence (referred to herein as “extension”). Polymerisation occurs in the methods described herein when the amplification solution is subjected to the polymerisation temperature. In other words, both hybridisation/annealing and extension are expected to occur when the amplification solution is subjected to the polymerisation temperature.

Polymerisation may occur following the hybridisation of an amplification primer to the nucleotide sequence at the 3′ end of the target nucleic acid. For example, polymerisation may occur following the hybridisation of the forward amplification primer to the nucleotide sequence at the 3′ end of a first strand of a double stranded target nucleic acid and/or the hybridisation of the reverse amplification primer to the 3′ end of a second strand of the target nucleic acid.

The amplification solution is subjected to a denaturation temperature to denature nucleic acid in the amplification solution. This may include for example, target nucleic acid, sample nucleic acid, primers and probes. The denaturation temperature may be around 85° C. or less, around 84° C. or less, around 83° C. or less, around 82° C. or less, around 81° C. or less, around 80° C. or less, around 79° C. or less, around 78° C. or less, around 77° C. or less, around 76° C. or less, or around 75° C. or less. Preferably, the denaturation temperature is 85° C. or less, 84° C. or less, 83° C. or less, 82° C. or less, 81° C. or less, 80° C. or less, 79° C. or less, 78° C. or less, 77° C. or less, 76° C. or less, or 75° C. or less. More preferably, the denaturation temperature is around 82° C. or less, for example the denaturation temperature may be 82° C. or less. Most preferably the denaturation temperature is around 81° C. or around 80° C., for example the denaturation temperature may be 81° C. or 80° C. A “low” denaturation temperature is, for example, a denaturation temperature of less than or equal to around 85° C.

The polymerisation temperature is the temperature to which the amplification solution is subjected in order for polymerisation to occur, i.e. for the “hybridisation” or “annealing” step and the “extension” step to occur. In other words, the polymerisation temperature is the temperature to which the amplification solution is subjected such that the primers hybridise to the target nucleic acid and are extended by the polymerase. Preferably, the polymerisation temperature is around 45° C. or less, around 44° C. or less, around 43° C. or less, around 42° C. or less, around 41° C. or less, around 40° C. or less, around 39° C. or less, around 38° C. or less, around 37° C. or less, around 36° C. or less, around 35° C. or less, around 34° C. or less, around 33° C. or less, around 32° C. or less, around 31° C. or less, around 30° C. or less, around 29° C. or less, around 28° C. or less, around 27° C. or less, around 26° C. or less, or around 25° C. or less. For example, the polymerisation temperature may be 45° C. or less, 44° C. or less, 43° C. or less, 42° C. or less, 41° C. or less, 40° C. or less, 39° C. or less, 38° C. or less, 37° C. or less, 36° C. or less, or 35° C. or less, 34° C. or less, 33° C. or less, 32° C. or less, 31° C. or less, 30° C. or less, 29° C. or less, 28° C. or less, 27° C. or less, 26° C. or less, or 25° C. or less. More preferably, the polymerisation temperature may be between around 30° C. and around 45° C., around 30° C. and around 44° C., around 30° C. and around 43° C., around 30° C. and around 42° C., around 30° C. and around 41° C., around 30° C. and around 40° C., around 30° C. and around 39° C., around 30° C. and around 38° C., around 30° C. and around 37° C., around 30° C. and around 36° C., around 30° C. and around 35° C., around 30° C. and around 34° C., around 30° C. and around 33° C., around 30° C. and around 32° C., or around 30° C. and around 31° C. For example, the polymerisation temperature may be between 30° C. and 45° C., 30° C. and 44° C., 30° C. and 43° C., 30° C. and 42° C., 30° C. and 41° C., 30° C. and 40° C., 30° C. and 39° C., 30° C. and 38° C., 30° C. and 37° C., 30° C. and 36° C., 30° C. and 35° C., 30° C. and 34° C., 30° C. and 33° C., 30° C. and 32° C., or 30° C. and 31° C. Most preferably, the polymerisation temperature is around 30° C., for example the polymerisation temperature may be 30° C. A “low” polymerisation temperature is, for example, a polymerisation temperature of less than or equal to around 45° C.

The amplification solution may be subjected to a denaturation temperature by placing the amplification solution in an environment at the denaturation temperature. In the denaturation step, the amplification vessel is exposed to an environment at the denaturation temperature, thereby subjecting the amplification solution to the denaturation temperature.

The amplification solution may be subjected to a polymerisation temperature by placing the amplification solution in an environment at the polymerisation temperature. In the polymerisation step, the amplification vessel is exposed to an environment at the polymerisation temperature, thereby subjecting the amplification solution to the polymerisation temperature.

The amplification solution may be subjected to the denaturation temperature for around 1.5 second or less, around 1.4 second or less, around 1.3 second or less, around 1.2 second or less, around 1.1 second or less, around 1.0 second or less, around 0.9 second or less, around 0.8 second or less, around 0.7 second or less, around 0.6 second or less, around 0.5 second or less, around 0.4 second or less, around 0.3 second or less, around 0.2 second or less, around 0.1 second or less, or around 0.0 second. Preferably, the amplification solution may be subjected to the denaturation temperature for 1.5 second or less, 1.4 second or less, 1.3 second or less, 1.2 second or less, 1.1 second or less, 1.0 second or less, 0.9 second or less, 0.8 second or less, 0.7 second or less, 0.6 second or less, 0.5 second or less, 0.4 second or less, 0.3 second or less, 0.2 second or less, 0.1 second or less, or 0.0 second. More preferably, the amplification solution may be subjected to the denaturation temperature for 1.0 second or less, 0.9 second or less, 0.8 second or less, 0.7 second or less, 0.6 second or less, 0.5 second or less, 0.4 second or less, 0.3 second or less, 0.2 second or less, 0.1 second or less, or 0.0 second. A “short” time period for subjecting the amplification solution to the denaturation temperature is, for example, for less than or equal to around 1.5 second.

The amplification solution may be subjected to the polymerisation temperature for around 1.0 second or less, around 0.9 second or less, around 0.8 second or less, around 0.7 second or less, around 0.6 second or less, around 0.5 second or less, around 0.4 second or less, around 0.3 second or less, around 0.2 second or less, around 0.1 second or less, or around 0.0 second. Preferably, the amplification solution may be subjected to the polymerisation temperature for 1.0 second or less, 0.9 second or less, 0.8 second or less, 0.7 second or less, 0.6 second or less, 0.5 second or less, 0.4 second or less, 0.3 second or less, 0.2 second or less, 0.1 second or less, or 0.0 second. More preferably, the amplification solution may be subjected to the polymerisation temperature for 0.5 second or less, 0.4 second or less, 0.3 second or less, 0.2 second or less, 0.1 second or less, or 0.0 second. A “short” time period for subjecting the amplification solution to the polymerisation temperature may include, for example, for less than or equal to around 1.0 second.

Without being bound by theory, the 3 conceptual stages of PCR (denaturation, primer annealing and polymerase extension) may not exclusively occur at certain “optimal” temperature. Rather, the rates at which denaturation, primer annealing and polymerase extension occur may vary with reaction temperature. Although each stage may be most efficient at a given “optimal” temperature (which depends upon, inter alia, the design of the amplicon and/or primer), the stage will still occur at temperatures surrounding that given temperature. For this reason, each conceptual stage can occur mostly during the temperature transitions before and after subjecting the amplification solution to the given “optimal” temperature for a very short length of time (e.g. less than 1 second or even 0 seconds).

Preferably, in the methods described herein, the amplification solution is thermally cycled between a denaturation region held at the denaturation temperature and a polymerisation region held at the polymerisation temperature. Repetition of the denaturation step and polymerisation step permits amplification of the target nucleic acid. As used herein, one “amplification cycle” refers to one denaturation step followed by one polymerisation step.

Preferably, each repetition of the denaturation step and polymerisation step is completed in 2 to 4 seconds, 2.5 to 4 seconds, 3 to 4 seconds, or 3.5 to 4 seconds. More preferably, each repetition of the denaturation step and polymerisation step may be completed in 2 to 4 seconds, 2 to 3.5 seconds, 2 to 3 seconds, or 2 to 2.5 seconds. Most preferably, each repetition of the denaturation step and polymerisation step may be completed in 4 seconds or less, 3.5 seconds or less, 3 seconds or less, 2.5 seconds or less, 2 seconds or less, or 1.5 seconds or less. A “short” time period for completing each repetition of the denaturation step and polymerisation step may include, for example, less than or equal to 4 seconds.

Preferably, the denaturation step and polymerisation steps are repeated 5 or more times, 10 or more times, 15 or more times, 20 or more times, 25 or more times or 30 or more times. More preferably, the denaturation step and polymerisation steps are repeated 30 to 40 times. For example, the denaturation step and polymerisation steps may be repeated 30 times.

Preferably, the repetitions of the denaturation step and polymerisation step to amplify the target nucleic acid are performed in 2 minutes or less, 1.75 minutes or less, 1.5 minutes or less, 1.25 minutes or less, or 1.0 minute or less. More preferably, the repetitions of the denaturation step and polymerisation step to amplify the target nucleic acid are performed in 1 minute to 2 minutes.

The amplification method disclosed herein may be preceded by an initial denaturation step whereby the amplification solution is subjected to an initial denaturation temperature. The initial denaturation temperature may be the same as, higher than, or lower than the denaturation temperature to which the amplification solution is subjected during each amplification cycle. Preferably, the initial denaturation temperature is the same as the denaturation temperature to which the amplification solution is subjected during each amplification cycle. This allows amplification method disclosed herein to be performed on a device having only one polymerisation region and one denaturation region without needing to ramp the temperature of the denaturation region up or down between the initial denaturation step and the start of the first amplification cycle, thereby allowing rapid amplification and detection of a target nucleic acid. Where an initial denaturation temperature is used which is higher or lower than the denaturation temperature to which the amplification solution is subjected during each amplification cycle, such rapid amplification and detection of a target nucleic acid can be maintained by using a device having one or more denaturation regions which can be held at different temperatures.

The length of time for which the amplification solution is subjected to an initial denaturation temperature may be 5 minutes or less, 4 minutes or less, 3 minutes or less, 2.5 minutes or less, 2 minutes or less, 1.5 minutes or less, 1 minute or less, 45 seconds or less, 30 seconds or less, 15 seconds or less, 10 seconds or less, 5 seconds or less, 4 seconds or less, 3 seconds or less, 2 seconds or less, 1 second or less, 0.75 seconds or less, 0.5 seconds or less, or 0.25 seconds or less. Preferably, the amplification solution is subjected to an initial denaturation temperature for 3 minutes.

A method described herein may further comprise an initial reverse transcription step in which a target ribonucleic acid is reverse transcribed to produce a target nucleic acid. The resulting target nucleic acid may comprise an RNA strand (corresponding to the target ribonucleic acid) and a DNA strand (corresponding to the reverse transcription-produced complement of the target ribonucleic acid) or may comprise two DNA strands (i.e. double stranded cDNA). The amplification method may comprise an initial reverse transcription step in which a target ribonucleic acid is reverse transcribed to produce the target nucleic acid.

The target ribonucleic acid may be present within a sample ribonucleic acid. The sample ribonucleic acid may comprise the target ribonucleic acid and a flanking portions or flanking portions of ribonucleic acid. For example, the target ribonucleic acid may be located within the sample ribonucleic acid. The sample ribonucleic acid may be, for example, an mRNA comprising the target ribonucleic acid or an RNA genome of a virus comprising the target ribonucleic acid.

The sample ribonucleic acid may be present in a sample previously obtained from an individual. The sample obtained from an individual may be, for example, a bodily fluid such as saliva, mucus, or blood.

Preferably, the target ribonucleic acid is transcribed by subjecting a sample comprising the target ribonucleic acid to a reverse transcription temperature of between 20° C. to 25° C. for between 1 minute to 10 minutes in the presence of a reverse transcriptase under suitable conditions. Suitable conditions may include the presence of nucleotides, a non-specific primer, such as poly(T), and an RNAse inhibitor. For example, the target ribonucleic acid may be transcribed by subjecting a sample comprising the target ribonucleic acid and a reverse transcriptase under suitable conditions to a reverse transcription temperature of 21° C. for between 1 minute to 10 minutes, by subjecting a sample comprising the target ribonucleic acid and a reverse transcriptase to a reverse transcription temperature of between 20° C. to 25° C. for 5 minutes or by subjecting a sample comprising the target ribonucleic acid and a reverse transcriptase to a reverse transcription temperature of 21° C. for 5 minutes.

Preferably, the sample comprising the target ribonucleic acid and a reverse transcriptase is subjected to a reverse transcription temperature of between around 20° C. to around 25° C., around 20° C. to around 24° C., around 20° C. to around 23° C., around 20° C. to around 22° C., around 20° C. to around 21° C., around 21° C. to around 25° C., around 21° C. to around 24° C., around 21° C. to around 23° C., or around 21° C. to around 22° C. For example, the sample comprising the target ribonucleic acid and a reverse transcriptase may be subjected to a reverse transcription temperature of between 20° C. to 25° C., 20° C. to 24° C., 20° C. to 23° C., 20° C. to 22° C., 20° C. to 21° C., 21° C. to 25° C., 21° C. to 24° C., 21° C. to 23° C., or 21° C. to 22° C. More preferably, the reverse transcriptase temperature may be around 20° C., around 21° C., around 22° C., around 23° C., around 24° C., or around 25° C. For example, the reverse transcriptase temperature may be 20° C., 21° C., 22° C., 23° C., 24° C., or 25° C. Even more preferably, the reverse transcription temperature is around room temperature or room temperature. Most preferably, the reverse transcription temperature is around 21° C., for example the reverse transcription temperature is 21° C.

Preferably, the sample comprising the sample ribonucleic acid and a reverse transcriptase is subjected to the reverse transcription temperature for 1 minute to 10 minutes. More preferably, the sample comprising the sample ribonucleic acid and a reverse transcriptase may be subjected to the reverse transcription temperature for around 1 minute, around 2 minutes, around 3 minutes, around 4 minutes, around 5 minutes, around 6 minutes, around 7 minutes, around 8 minutes, around 9 minutes, or around 10 minutes. For example, the sample comprising the sample ribonucleic acid and a reverse transcriptase may be subjected to the reverse transcription temperature for 1 minute, 2 minutes, 3 minutes, 4 minutes, 5 minutes, 6 minutes, 7 minutes, 8 minutes, 9 minutes, or 10 minutes. More preferably, the sample comprising the sample ribonucleic acid and a reverse transcriptase is subjected to the reverse transcription temperature for around 4 minutes, 5 minutes, or 6 minutes, for example for 4 minutes, 5 minutes or 6 minutes. Most preferably the sample comprising the sample ribonucleic acid and a reverse transcriptase is subjected to the reverse transcription temperature for around 5 minutes, for example for 5 minutes.

The target ribonucleic acid may be present within a sample ribonucleic acid, such that, in an initial reverse transcription step, a sample ribonucleic acid comprising a target ribonucleic acid is reverse transcribed to produce a sample nucleic acid comprising the target nucleic acid.

Also provided is a nucleic acid amplification device suitable for use in the amplification methods described herein. A nucleic acid amplification device may comprise;
a denaturation region,
a polymerisation region,
a first heater to heat the denaturation region to between 75° C.-85° C.,
a second heater to heat the polymerisation region to between 25° C.-45° C., and
an actuator to move an amplification vessel containing an amplification solution between the denaturation region and the polymerisation region.

The nucleic acid amplification device may further comprise a processor programmed to operate the device to amplify a target nucleic acid by the amplification methods described herein.

A denaturation region is a volume which can be heated to the denaturation temperature. Preferably, the denaturation region is a water bath.

A polymerisation region is a volume which can be heated to the polymerisation temperature. Preferably, the polymerisation region is a water bath.

The temperatures of the denaturation region and polymerisation region are raised to the denaturation and polymerisation temperatures using a heater. For example, a first heater may be used to raise the temperature of the denaturation region to the denaturation temperature and a second heater may be used to raise the temperature of the polymerisation region to the polymerisation temperature.

The amplification solution may be contained in an amplification vessel. The amplification reaction occurs in the amplification solution in the amplification vessel. Suitable amplification vessels include preferably a plate, a tube, or a container. Suitable amplification vessels have preferably a high thermal conductivity; for example, preferably, the amplification vessel is formed of plastic, glass or metal.

The amplification vessel preferably has a total volume of 10 μL or less, 9 μL or less, 8 μL or less, 7 μL or less, 6 μL or less, 5 μL or less, 4.5 μL or less, 4 μL or less, 3.5 μL or less, 3 μL or less, 2.5 μL or less, 2 μL or less, 1.5 μL or less, 1.0 μL or less, or 0.5 μL or less. More preferably, the amplification vessel has a total volume of 5 μL or less, 4.5 μL or less, 4 μL or less, 3.5 μL or less, 3 μL or less, 2.5 μL or less, 2 μL or less, 1.5 μL or less, 1.0 μL or less, or 0.5 μL or less. In some preferred embodiments, for example for microfluidics systems, the amplification vessel most preferably has a total volume of 2 μL or less, 1.5 μL or less, 1.0 μL or less, or 0.5 μL or less. The amplification is necessarily equal to or larger than the total volume of the amplification solution. Preferably, the amplification vessel has a volume that is 2 times that of the total volume of the amplification solution or 1.5 times that of the total volume of the amplification solution.

The actuator moves the amplification vessel between the denaturation region and the polymerisation region. Preferably, the actuator is a robotic arm.

Movement of the amplification vessel between the denaturation region, heated to the denaturation temperature, and the polymerisation region, heated to the polymerisation temperature, by the actuator subjects the amplification solution to the denaturation temperature such that the nucleic acid in the amplification solution is denatured and to the polymerisation temperature such that the primers hybridise to the target nucleic acid and are extended by the polymerase.

Preferably, the actuator is configured to move the amplification vessel from the denaturation region to the polymerisation region or from the polymerisation region to the denaturation region in 2 seconds or less, 1.75 seconds or less, 1.5 seconds or less, 1.25 seconds or less, 1.0 seconds or less, 0.75 seconds or less, 0.5 seconds or less, 0.25 seconds or less. More preferably, the actuator is configured to move the amplification vessel from the denaturation region to the polymerisation region or from the polymerisation region to the denaturation region in 1.0 seconds or less, 0.75 seconds or less, 0.5 seconds or less, 0.25 seconds or less.

The processor is an electronic circuit which executes programmed instructions. The term “programmed to operate” means that the processor is provided with the instructions for performing a certain operation. The processor referred to herein is programmed to operate the device of the present invention to amplify a target nucleic acid by the methods of the present invention. This includes, for example, instructions to heat the denaturation region and polymerisation region to a predetermined denaturation temperature and polymerisation temperature, respectively and to hold the amplification vessel in the denaturation region and the polymerisation region for predetermined time periods.

The device may be configured to adjust the denaturation temperature and/or the polymerisation temperature. Preferably, the device is configured to adjust the denaturation temperature and/or the polymerisation temperature within the ranges for the denaturation temperature and/or polymerisation temperature discussed above. It may be desirable to adjust polymerisation temperature according to the lengths, sequences and melting temperatures of the forward and reverse amplification primers and to adjust denaturation temperature according to the length, sequence and melting temperature of the target nucleic acid. Different target nucleic acids may differ in length, sequence and melting temperature and will require different forward and reverse amplification primers which may differ in length, sequence and melting temperature to those used for another target nucleic acid. Accordingly, amplification reactions for amplifying different target nucleic acids may require different denaturation temperatures and/or polymerisation temperatures. A device configured to adjust the denaturation temperature and/or the polymerisation temperature can therefore be used for amplification reactions amplifying different target nucleic acids.

The device may be configured to adjust the length of time for which the amplification solution is subjected to the denaturation temperature and/or polymerisation temperature. Preferably, the device is configured to adjust the length of time for which the amplification solution is subjected to the denaturation temperature and/or polymerisation temperature within the ranges for the length of time for which the amplification solution is subjected to the denaturation temperature and/or polymerisation temperature disclosed above. The length of time for which it is necessary to subject the amplification solution to the denaturation temperature and/or polymerisation temperature depends upon a number of factors including, but not limited to: the length, secondary structure, and melting temperature of the target nucleic acid; the concentration and melting point of the forward and reverse amplification primers; the heat conductivity of the reaction vessel; and the volume of the amplification solution. In order to achieve the fastest amplification reaction possible, it is desirable to minimise the length of time for which the amplification solution is subjected to the denaturation temperature and/or polymerisation temperature. It will be appreciated that many of the factors discussed will differ depending on the choice of target nucleic acid. Accordingly, a device configured to adjust the length of time for which the amplification solution is subjected to the denaturation temperature and/or polymerisation temperature can be used for amplification reactions amplifying different target nucleic acids. In addition, such a device can be used to perform amplification reactions amplifying the same target nucleic acid under different conditions, whereby the use of different conditions alters the length of time for which it is necessary to subject the amplification solution to the denaturation temperature and/or polymerisation temperature.

Preferably, the device is configured to adjust the length of time taken for the actuator to move the amplification vessel from the denaturation region to the polymerisation region or from the polymerisation region to the denaturation region. In other words, preferably the device is configured to adjust the speed at which the actuator moves the amplification vessel from the denaturation region to the polymerisation region or from the polymerisation region to the denaturation region

Also provided is a system for amplifying a target nucleic acid comprising:
(i) the nucleic amplification device described above,
(ii) a forward amplification primer and a reverse amplification primer which are specific for the target nucleic acid;
(iii) deoxyribonucleotide triphosphates and;
(iv) a DNA polymerase.

The device of the present invention is as described above. Suitable target nucleic acids, forward amplification primers, reverse amplification primers, deoxyribonucleotide triphosphates and DNA polymerases are described above.

The reagents described above (including the forward and reverse amplification primers, deoxyribonucleotide triphosphates and DNA polymerase) may be present in a liquid solution or in a lyophilised form. When the reagents are provided in a liquid solution, the liquid solution may be an aqueous solution. A sterile aqueous solution is particularly preferred. Certain reagents may be provided as dried powder(s) which can be reconstituted by, for example, the addition of a suitable solvent.

The reagents described above are preferably provided in containers from which they can be transferred, preferably aliquoted, into the amplification vessel. Preferably, each reagent is provided in an individual container from which it can be transferred, preferably aliquoted, into the amplification vessel.

The reagent container is preferably sterile. The amplification vessel is preferably sterile.

The device may be accompanied by instructions for using the device, for example by instructions for using the device to amplify a target nucleic acid.

The preferred embodiments described above in relation to the methods and devices of the present invention apply equally to the systems of the present invention.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

It is to be understood that the application discloses all combinations of the ranges and/or parameters described above either singly or together with any of the other ranges and/or parameters described above, unless context demands otherwise. The application discloses all combinations of the ranges and/or parameters described above either singly or together with any of the aspects and embodiments described above, unless context demands otherwise. Similarly, the application discloses all combinations of the ranges and/or parameters described above either singly or together with any of the preferred and/or optional features, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

“around” where used herein is to be taken as specific disclosure of any value or range having the same round-off as the specified value or range. For example, “around 70° C.” is to be taken as specific disclosure of any value having the same round-off as the specified value, including, for example, 69.5° C. and 70.49° C. For example, “around 1.0 s” is to be taken as specific value as any value having the same round-off as the specified value, including, for example, 0.95 s and 1.049 s.

### EXPERIMENTAL

### Materials and Methods

### Exemplary PCR Protocol

The reaction mixture contains:
2× concentration of Taq polymerase,
primers at 10 μM and
probe at 100-200 nM final concentration.

The volume of the reaction mixture can be between 1 and 15 μL, but smaller volumes are achievable.

PCR is performed as a two-step reaction.

Under a first program, in the denaturation step, the reaction mixture is subjected to a temperature of around 80° C. for 1 second. In the subsequent polymerisation step, the reaction mixture is subjected to a temperature of 30° C. for less than 1 second. Cycling between two temperature zones eliminates the time it takes to ramp between temperatures, and so allows 30-40 cycles to be completed in around 1 minute.

Under a second program, in the denaturation step, the reaction mixture is subjected to a denaturation temperature of for 1.5 seconds. In the subsequent polymerisation step, the reaction mixture is subjected to an annealing/polymerisation temperature for 0 seconds. Using this program, a PCR run comprising 30 cycles can be completed in 75 seconds.

Optionally, a reverse transcription step can be added before the denaturation step. The reverse transcription reaction can be performed at room temperature (around 21° C.) for around 5 minutes before subjecting the reverse transcription reaction mixture to the exemplary PCR protocol described above. To perform the reverse transcription reaction, the reagents (including reverse transcriptase, deoxyribonucleotide triphosphates, primer, buffer and/or water) and sample RNA can be added into a reverse transcription vessel (for example, a tube) and left to stand at room temperature for around 5 minutes. Preferably, the reagents, sample RNA and reverse transcription vessel are kept at room temperature. After performing the reverse transcription reaction, the reverse transcription reaction mixture is preferably immediately subjected to the exemplary PCR protocol described above.

### Exemplary Extreme PCR Instrument

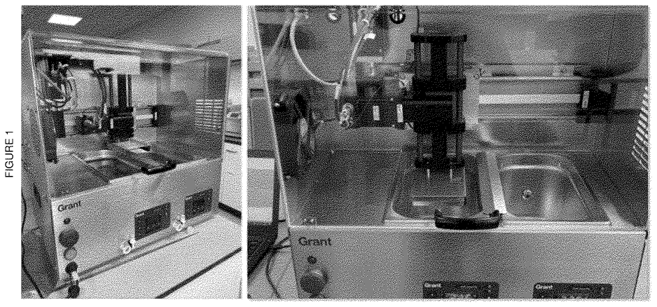

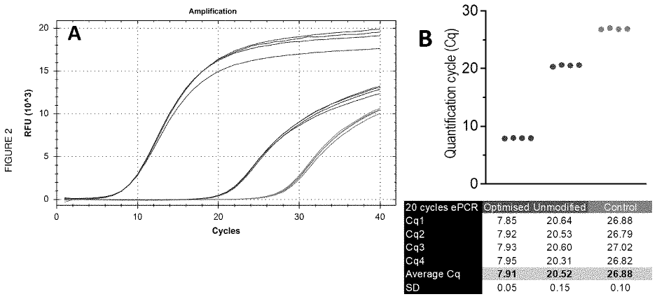

The extreme PCR reactions described herein were performed on a bespoke water bath PCR instrument, as shown in FIG. 1. The instrument, also referred to herein as a “Mk1extreme PCR device”, contained two water baths. One water bath was kept at the denaturation temperature and the other at the polymerisation temperature. A robotic arm, capable of holding and moving plates or tubes containing the PCR reactions, cycled the PCR reactions (in their respective plate or tube) between the two water baths. The time for which the robotic arm holds plates or tubes containing the PCR reactions in each water bath can be adjusted from 0 seconds to an infinite length of time.

As compared to traditional PCR instruments, the MK1extreme PCR device permits a faster total reaction time because, during the extreme PCR reaction, each water bath of the MK1extreme PCR device represents a fixed temperature zone. Rather than spending time ramping the temperature between the annealing/polymerisation and denaturation temperatures, the PCR reactions (in their respective plate or tube) are simply transferred between the two water baths.

The above-described PCR instrument allows adjustment of the speed of movement of the robotic arm as well as of the dwelling time of the PCR reactions (in their respective plate or tube) in each of the denaturation water bath and polymerisation water bath.

At its fastest speed, the present inventors have found that the PCR instrument can complete 30 cycles of denaturation and polymerisation in 1 minute. The Mk1extreme PCR device eliminates the time it takes to ramp between temperatures on conventional PCR instruments.

The present inventors have efficiently extracted and enriched viral RNA and synthesised cDNA from saliva samples in around 5 minutes using the Mk1extreme PCR device.

### Results

### Example 1: Proof-of-Concept of the Extreme PCR Method and Effect of Optimising the Amplicon and Primers

qPCR was performed on the exemplary extreme PCR instrument to quantify the DNA in 3 different samples, each originating from Sample A, which comprised a target nucleic acid. A 0.5 μL aliquot of each sample was then added to 1×qPCR master mix and reamplified.

The 3 samples used for qPCR were as follows:
Test (brown): an amplified reaction mixture obtained from a 20-cycle amplification of Sample A performed on the exemplary extreme PCR instrument with optimised assay conditions in which the final concentration of each primer was 10 μM and the final concentration of Taq polymerase of 50 U/μL (2× the “conventional” concentration of 25 U/μL).
Control 1 (blue): an amplified reaction mixture obtained from 20-cycle amplification of Sample A performed on the Mk1extreme PCR device using non-optimised “conventional” assay conditions in which the final concentration of each primer was 500 nM and the final enzyme concentration was 25 U/μL.
Control 2 (red): a reaction mixture obtained from Sample A in which each primer and the enzyme were added at a final concentration of 500 nM and 25 U/μL, respectively, but which was not subjected to 20 cycles of amplification on the Mk1extreme PCR device.

The results of this experiment are shown in FIG. 2. The average Cq values for both the Test (amplified using optimised amplicon and primers) and Control 1 (non-optimised assay conditions) mixtures were lower than the average Cq value for Control 2 (no prior amplification). The present inventors have therefore shown that the extreme PCR method described herein effectively amplifies DNA.

In addition, the average Cq value for Test (amplified using optimised amplicon and primers) mixture was significantly lower than for Control 1 (non-optimised assay conditions) mixtures. The present inventors have therefore shown that the optimisation of amplicons and primers greatly enhances the effectiveness of the extreme PCR method at amplifying DNA.

### Example 2: Reproducibility of Extreme PCR

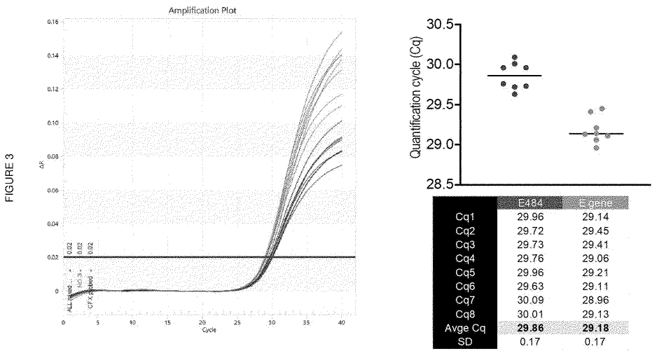

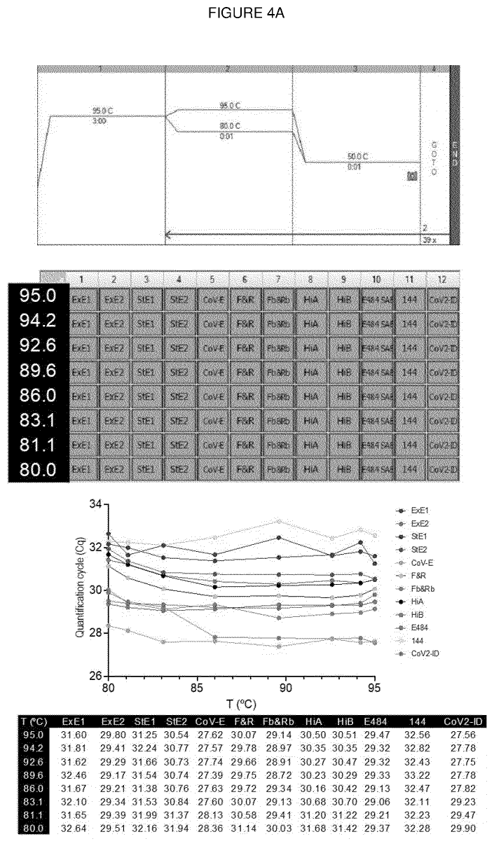

8 replicate reactions probing for the E484 WT sequence (shown in blue on the amplification plot of FIG. 3) using optimised amplicons were amplified for 10 cycles on the exemplary extreme PCR instrument.

In addition, 8 replicate reactions probing for the E gene (shown in green on the amplification plot of FIG. 3) using optimised amplicons were amplified for 10 cycles on the exemplary extreme PCR instrument.

Subsequently, 0.5 μL aliquots of each replicate reaction were diluted 1:1000 in water and run on a standard qPCR instrument.

The results of this experiment are shown in FIG. 3. The maximum Cq range between replicates was 0.46 for the E484 WT sequence (Cq7: 30.09-Cq6: 29.63) and 0.49 for the E-gene (Cq2: 29.45-Cq7: 28.96). In other words, the maximum Cq range between replicates for both the E484 WT sequence and the E-gene were <0.5. In addition, the standard deviations between the 8 replicate reactions were small for both the E484 WT sequence and the E-gene.

Accordingly, the present inventors have shown that the extreme PCR methodology described herein is highly reproducible.

### Example 3: Effect of Reduced Denaturation Temperature on Different PCR Amplicons

### Example 3A: Denaturation Temperatures Between 80° C. and 95° C.

PCR was performed on a standard qPCR instrument on 12 different amplification solutions, as follows:
1. Subjecting each amplification solution to 95.0° C. for 3.00 s (initial denaturation)
2. Subjecting each amplification solution to between 80.0° C. to 95.0° C. for 0.01 s (denaturation)
3. Subjecting each amplification solution to at 50.0° C. for 0.01 s (polymerisation)
4. Repeating steps 2-3 such that steps 2-3 are completed a total of 39 times.

The 12 different amplification solutions targeted: ExE1, ExE2, StE1, StE2, Cov-E, F&R, Fb&Rb, HiA, HiB, E484, 144 and CoV2-ID.

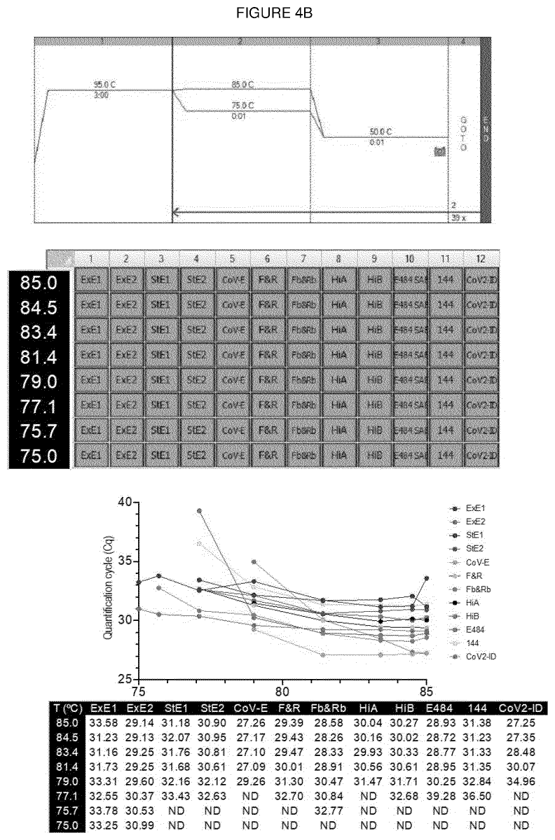

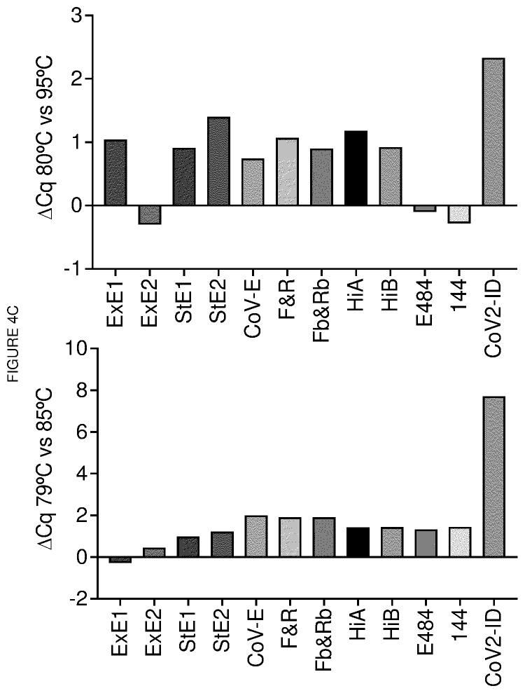

The results of this experiment are shown in FIG. 4 (left-hand panel and the upper graph in the right-hand panel). All PCR amplicons showed highly efficient template denaturation from 95° C. down to 80° C. For 11 targets, the Cq range between 80° C. and 95° C. was less than 2. For the CoV2-ID, which was not designed for extreme PCR, the maximum Cq range between 80° C. and 95° C. was less than 3.

Accordingly, the present inventors have shown that it is possible to achieve efficient template denaturation down to around 80° C.

### Example 3B: Denaturation Temperatures Between 75° C. and 95° C.

PCR was performed on a standard qPCR instrument for 12 amplification solutions, as follows:
1. Subjecting each amplification solution to 95.0° C. for 3.00 s (initial denaturation)
2. Subjecting each amplification solution to between 75.0° C. to 95.0° C. for 0.01 s (denaturation)
3. Subjecting each amplification solution to at 50.0° C. for 0.01 s (polymerisation)
4. Repeating steps 2-3 such that steps 2-3 are completed a total of 39 times.

As for Example 3A, the 12 different amplification solutions targeted: ExE1, ExE2, StE1, StE2, Cov-E, F&R, Fb&Rb, HiA, HiB, E484, 144 and CoV2-ID.

The results of this experiment are shown in FIG. 4 (centre panel and the lower graph in the right-hand panel). Many PCR amplicons showed efficient template denaturation below 80° C.; the Cq range between 79° C. and 85° C. was less than 3 for 11 of the 12 targets. As in Example 3A, the CoV2-ID target showed a relatively high Cq range. Some targets, such as ExE1 and Exe2, showed efficient template denaturation down to 75° C.

Accordingly, the present inventors have shown that it is possible to achieve efficient template denaturation down to 79° C.

### Example 3C: Confirmation of Adequate Assay Performance at Around 80° C.

The inventors then performed another experiment to confirm that adequate assay performance could be achieved at these low temperatures.

PCR was performed on a standard qPCR instrument for 12 amplification solutions, as follows:
1. Subjecting each amplification solution to 95.0° C. for 3.00 s (initial denaturation)
2. Subjecting each amplification solution to between 65.0° C. to 80.5° C. for 0.01 s (denaturation)
3. Subjecting each amplification solution to at 50.0° C. for 0.01 s (polymerisation)
4. Repeating steps 2-3 such that steps 2-3 are completed a total of 39 times.

As for Examples 3A and 3B, the 12 different amplification solutions targeted: ExE1, ExE2, StE1, StE2, Cov-E, F&R, Fb&Rb, HiA, HiB, E484, 144 and CoV2-ID.

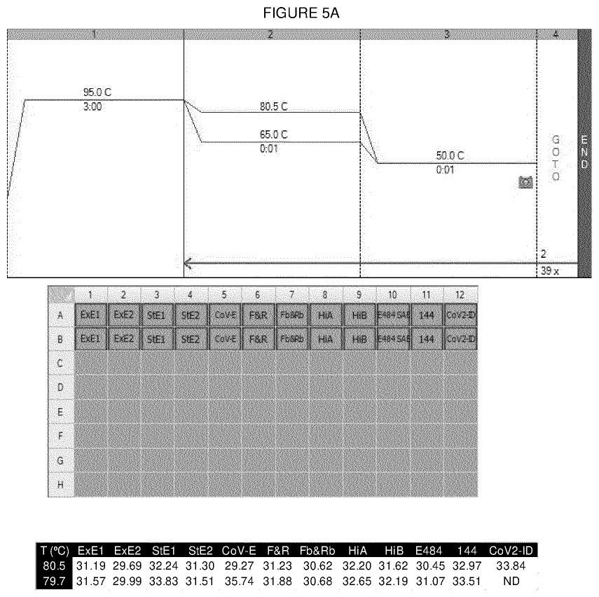

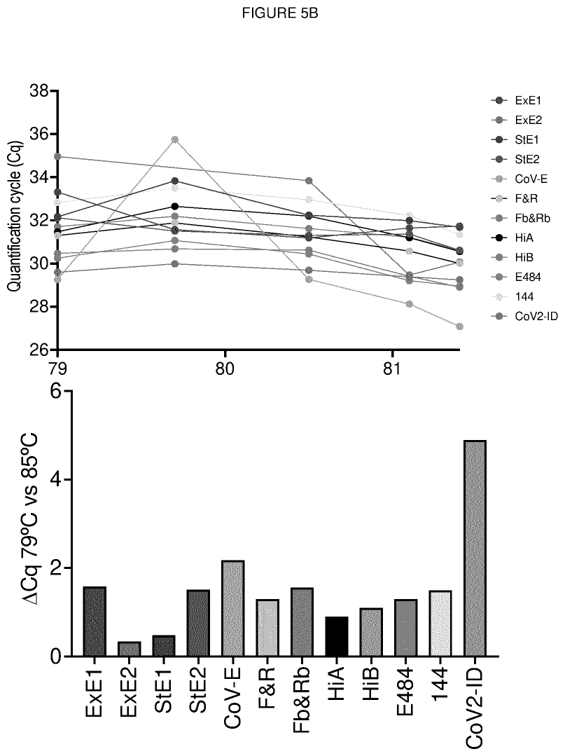

The results of this experiment are shown in FIG. 5. Many PCR amplicons showed efficient template denaturation below 80° C.; the Cq range between 79° C. and 85° C. was less than 3 for 11 of the 12 targets. As in Examples 3A and B, the CoV2-ID target showed a relatively high Cq range.

These results are concordant with those of Examples 3A and 3B. Accordingly, the present inventors have shown that it is possible to reliably achieve adequate denaturation, and therefore adequate assay performance, down to 79° C. In addition, the present inventors have shown that amplicon design is an essential component of successful performance.

### Example 4: Effect of Reduced Polymerisation Temperature on Different PCR Amplicons

PCR was performed on a standard qPCR instrument on 12 different amplification solutions, as follows:
1. Subjecting each amplification solution to 95.0° C. for 3.00 s (initial denaturation)
2. Subjecting each amplification solution to 95.0° C. for 0.01 s (denaturation)
3. Subjecting each amplification solution to between 30.0° C. to 54.0° C. for 0.01 s (polymerisation)
4. Repeating steps 2-3 such that steps 2-3 are completed a total of 39 times.

As for Examples 3A-3C, the 12 different amplification solutions targeted: ExE1, ExE2, StE1, StE2, Cov-E, F&R, Fb&Rb, HiA, HiB, E484, 144 and CoV2-ID.

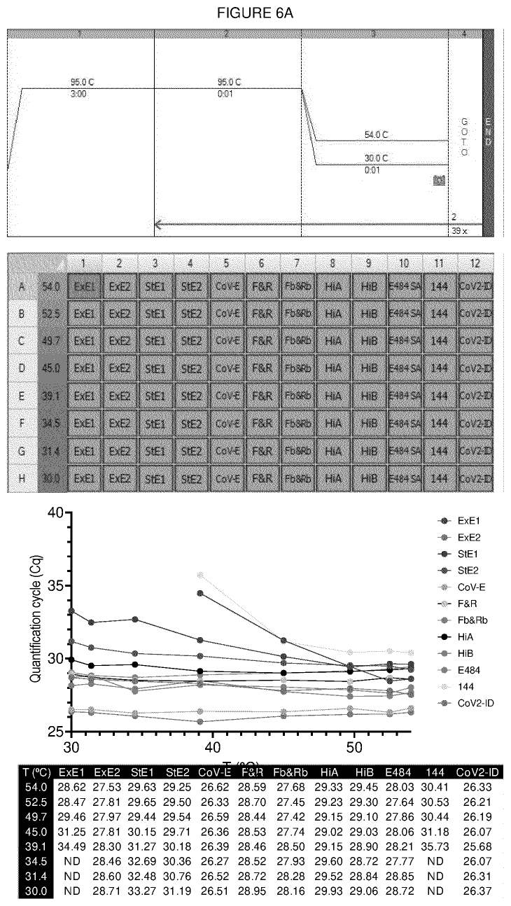

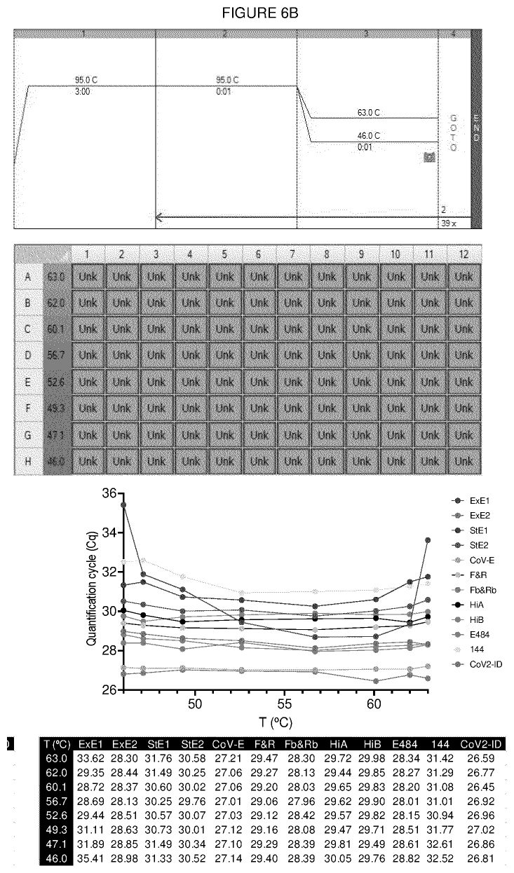

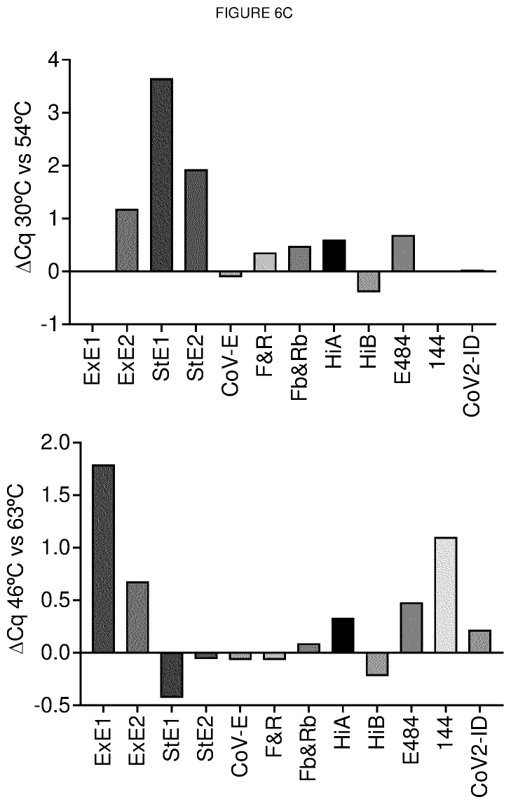

The results of this experiment are shown in FIG. 6. Many PCR amplicons were efficiently amplified at low temperatures. Dependent on the PCR primers and amplicons, the polymerisation can be as efficient at 30° C. as at 63° C. (see, for example, CoV-E). Some PCR amplicons were particularly sensitive to low temperatures, such as ExE1 and 144.

The above shows that efficient amplification may be achieved at temperatures as low as 30° C.

### REFERENCES

1. 1. Wheeler, E. K., Hara, C. A., Frank, J., Deotte, J., Hall, S. B., Benett, W., Spadaccini, C. and Beer, N. R. (2011). Under-three minute PCR: Probing the limits of fast amplification. Analyst, [online] 136(18), pp. 3707-3712. Available at:
2. Farrar, J. S. and Wittwer, C. T. (2015). Extreme PCR: Efficient and Specific DNA Amplification in 15-60 Seconds. Clinical Chemistry, 61(1), pp. 145-153.

1. A method for amplifying a target nucleic acid comprising:
(i) contacting a target nucleic acid with a forward and reverse amplification primers specific for the target nucleic acid in the presence of DNA polymerase and deoxyribonucleotide triphosphates to produce an amplification solution,
wherein the amplification solution has a volume of 5 μl or less,
(ii) subjecting the amplification solution to a denaturation temperature of 75° C.-85° C. for 1.5 second or less, such that nucleic acid in the amplification solution is denatured,
(iii) subjecting the amplification solution to a polymerisation temperature of 25° C.-45° C. for 1 second or less,
such that the primers hybridise to the target nucleic acid and are extended by the polymerase,
(iv) repeating steps (ii) and (iii) one or more times, thereby amplifying the target nucleic acid.

2. The method of claim 1, wherein each repeat of steps (ii) and (iii) is completed in 4 seconds or less.

3. The method of claim 2, wherein each repeat of steps (ii) and (iii) is completed in 2-4 seconds.

4. The method of any preceding claim, wherein steps (ii) and (iii) are repeated 5 or more times, 10 or more times, 15 or more times, 20 or more times, 25 or more times or 30 or more times.

5. The method of claim 4, wherein steps (ii) and (iii) are repeated at least 30 times.

6. The method of claim 4 or 5, wherein steps (ii) and (iii) are repeated 30-40 times.

7. The method of any preceding claim, wherein steps (ii) to (iv) are performed in 2 or less minutes to amplify the target nucleic acid.

8. The method of claim 7, wherein steps (ii) to (iv) are performed in 1-2 minutes to amplify the target nucleic acid.

9. The method of any preceding claim, wherein the forward and reverse amplification primers have a melting temperature of between 40° C.-50° C.

10. The method of claim 9, wherein the forward and reverse amplification primers have a melting temperature of 45° C.

11. The method of any preceding claim, wherein the target nucleic acid is 50-60 bases in length.

12. The method of any preceding claim, wherein the melting temperature of the target nucleic acid is around 70° C.

13. The method of any preceding claim, wherein the concentration of each of the forward amplification primer and reverse amplification primer in the amplification solution is between 5 μM and 15 μM.

14. The method of claim 13, wherein the concentration of each of the forward amplification primer and reverse amplification primer in the amplification solution is 10 μM

15. The method of any preceding claim, wherein the DNA polymerase is present at a concentration of 50 U/mL

16. The method of any preceding claim, wherein the DNA polymerase is a thermostable polymerase, preferably Taq polymerase.

17. The method of any preceding claim, wherein the volume of the amplification solution is 1 μL or less

18. The method of claim 17, wherein the volume of the amplification solution is 0.5 μL or less.

19. The method of any preceding claim, wherein the amplification solution further comprises a probe to determine the amount of amplified target nucleic acid.

20. The method of claim 19, wherein the concentration of the probe in the amplification solution is between 100 nM to 200 nM.

21. The method of any preceding claim, wherein the amplification solution is thermally cycled between a denaturation region held at the denaturation temperature and a polymerisation region held at the polymerisation temperature.

22. The method of any preceding claim, wherein the amplification solution is substantially free of a PCR enhancer.

23. The method of claim 22, wherein the PCR enhancer is an organic compound.

24. The method of claim 23, wherein the organic compound is glycerol or ethylene glycol.

25. The method of any preceding claim, wherein the forward and reverse amplification primers are contacted with a sample nucleic acid that comprises the target nucleic acid.

26. The method of claim 25, wherein the target nucleic acid is located within a sample nucleic acid.

27. The method of any preceding claim, wherein the sample nucleic acid is a deoxyribonucleic acid produced by a method comprising providing a sample ribonucleic acid and reverse transcribing the sample ribonucleic acid to produce the sample nucleic acid.

28. The method of any preceding claim, wherein the sample ribonucleic acid is transcribed by subjecting the sample comprising a target ribonucleic acid and a reverse transcriptase to a reverse transcription temperature of between 20° C.-25° C. for 1-10 minutes.

29. The method of claim 28, wherein the reverse transcription temperature is room temperature.

30. The method of claim 28, wherein the reverse transcription temperature is 21° C.

31. The method of any one of claims 28-30, wherein the sample comprising a target nucleic acid and a reverse transcriptase is subjected to the reverse transcription temperature for 5 minutes.

32. A nucleic acid amplification device comprising;
a denaturation region,
a polymerisation region,
a heater to heat the denaturation region to between 75° C.-85° C.,
a heater to heat the polymerisation region to between 25° C.-45° C., and
an actuator to move an amplification vessel for containing an amplification solution between the denaturation region and the polymerisation region.

33. The device of claim 32, wherein the device further comprises a processor programmed to operate the device to amplify a target nucleic acid by a method any one of claims 1 to 31.

34. The device of claim 33, wherein the amplification vessel for containing an amplification solution has a total volume of 10 μL or less.

35. The device of claim 34, wherein the amplification vessel for containing an amplification solution has a total volume of 5 μL or less.

36. The device of claim 35, wherein the amplification vessel for containing an amplification solution has a total volume of 1 μL or less.

37. The device of any preceding claim, wherein the actuator to move the amplification vessel between the denaturation region and the polymerisation region is configured to move the amplification vessel from the denaturation region to the polymerisation region or from the polymerisation region to the denaturation region in 2 seconds or less.

38. The device of claim 37, wherein the actuator to move the amplification vessel between the denaturation region and the polymerisation region is configured to move the amplification vessel from the denaturation region to the polymerisation region or from the polymerisation region to the denaturation region in 1 second or less.

39. The device of any preceding claim, wherein the device is configured to adjust the length of time for which the amplification solution is subjected to the denaturation temperature and/or polymerisation temperature.

40. The device of any preceding claim, wherein the device is configured to adjust the denaturation temperature and/or polymerisation temperature.

41. The device of any preceding claim, wherein the device is configured to adjust the length of time taken for the actuator to move the amplification vessel from the denaturation region to the polymerisation region or from the polymerisation region to the denaturation region.

42. The device of any preceding claim, wherein the actuator to move the amplification vessel between the denaturation region and the polymerisation region is a robotic arm.

43. The device of any preceding claim, wherein the amplification vessel for containing an amplification solution is a plate or tube.

44. The device of any preceding claim, wherein the denaturation region and/or the polymerisation region is a water bath.

45. A system for amplifying a target nucleic acid comprising:
(i) a device according to any one of claims 32 to 44;
(ii) a forward amplification primer and a reverse amplification primer which are specific for the target nucleic acid;
(iii) deoxyribonucleotide triphosphates and;
(iv) a DNA polymerase.

46. The system of claim 45, wherein the forward and reverse amplification primers have a melting temperature of between 40° C.-50° C.

47. The system of claim 46, wherein the forward and reverse amplification primers have a melting temperature of 45° C.

48. The system of any preceding claim, wherein the target nucleic acid is 50-60 bases in length.

49. The system of any preceding claim, wherein the melting temperature of the target nucleic acid is around 70° C.

50. The system of any preceding claim, wherein the concentration of the forward amplification primer and reverse amplification primer in the amplification solution is between 5 μM and 15 μM.

51. The system of claim 50, wherein the concentration of the forward amplification primer and reverse amplification primer in the amplification solution is 10 μM

52. The system of any preceding claim, wherein the system further comprises a probe to determine the amount of amplified target nucleic acid.

53. The system of any preceding claim, wherein the final concentration of the probe is between 100 nM to 200 nM.

54. The system of any preceding claim, wherein the DNA polymerase is present at a concentration of 50 U/mL.

55. The system of any preceding claim, wherein the DNA polymerase is a thermostable polymerase, preferably Taq polymerase.
